(19) United States
(12) Patent Application Publication
(10) Pub. No.: US 2026/0024666 A1
(43) Pub. Date: Jan. 22, 2026

(54) DATA PROCESSING METHOD AND DEVICE, HEALTH ASSESSMENT METHOD AND DEVICE, ELECTRONIC DEVICE AND READABLE STORAGE MEDIUM

(71) Applicants: BEIJING BOE TECHNOLOGY DEVELOPMENT CO., LTD., Beijing (CN); BOE TECHNOLOGY GROUP CO., LTD., Beijing (CN)

(72) Inventors: Yude Li, Beijing (CN); Zhou Wang, Beijing (CN); Chunlin Song, Beijing (CN); Yiming Lu, Beijing (CN)

(73) Assignees: BEIJING BOE TECHNOLOGY DEVELOPMENT CO., LTD., Beijing (CN); BOE TECHNOLOGY GROUP CO., LTD., Beijing (CN)

(21) Appl. No.: 18/995,968

(22) PCT Filed: May 15, 2024
(86) PCT No.: PCT/CN2024/093315

§ 371 Date: Jan. 17, 2025

(30) Foreign Application Priority Data
Jun. 25, 2023 (CN) 202310752854.4

(51) Int. Cl. G16H 50/30; A61B 5/00; G16H 10/20

(52) U.S. Cl. CPC G16H 50/30; A61B 5/7267; G16H 10/20

(57) ABSTRACT

A data processing method and device, a health assessment method and device, an electronic device, and a readable storage medium are provided. The data processing method includes the following steps: acquiring the vital sign data of the target object, wherein the vital sign data includes monitoring data obtained by monitoring the vital signs of the target object and supplementary data for supplementing the vital sign data according to the monitoring data; collecting multimodal data of the target object, where the multimodal data includes at least one of the image data of the target object and the survey data for the preset symptoms; generating the physical state data of the target object according to the vital sign data and the multimodal data.

### CROSS-REFERENCE TO RELATED APPLICATION

The present application claims a priority to the Chinese patent application No. 202310752854.4 filed on Jun. 25, 2023, a disclosure of which is incorporated herein by reference in its entirety.

### TECHNICAL FIELD

The embodiments of the present disclosure relate to the technical field of disease diagnosis, and in particular, to a data processing method and device, a health assessment method and device, an electronic device, and a readable storage medium.

### BACKGROUND

With the development of computer technology, in related technologies, it is possible to collect user's physical data and analyze and predict the user's disease status or disease probability based on the collected results.

### SUMMARY

The embodiments of the present disclosure provide a data processing method and device, a health assessment method and device, an electronic device, and a readable storage medium.

In a first aspect, a data processing method is provided in the embodiment of the present disclosure, including:
acquiring vital sign data of a target object, where the vital sign data includes monitoring data obtained by monitoring the vital signs of the target object and supplemented data obtained by supplementing the vital sign data according to the monitoring data;
collecting multimodal data of the target object, where the multimodal data includes at least one of image data of the target object and survey data for preset symptoms; and
generating a physical state data of the target object according to the vital sign data and the multimodal data.

Optionally, the step of obtaining the vital sign data of the target object includes:
acquiring monitoring data obtained by monitoring the vital signs of the target object;
binning the monitoring data;
grouping the monitoring data after binning according to a preset monitoring period;
generating supplementary data to supplement the missing monitoring data in each monitoring cycle; and
using the monitoring data and the supplemented data as vital sign data of the target object.

Optionally, the binning the monitoring data includes:
binning the monitoring data according to the degree of impact of the monitoring data on the target disease.

Optionally, the binning the monitoring data includes:
binning the monitoring data using the minimum entropy binning method.

Optionally, after the monitoring data after binning is grouped according to a preset monitoring period, the method further includes:
detecting the number of first cycles in the monitoring cycle, where the first cycle is a monitoring cycle in which monitoring data at a target time is missing;
when the number of the first cycles is greater than a preset number threshold, determining the monitoring data at the target moment of the first cycle according to the monitoring data at the target moment of the second cycle, where the second cycle is a monitoring period in which the monitoring data at the target moment is not missing.

Optionally, generating the supplementary data for supplementing the missing monitoring data in each monitoring cycle includes:
generating the complementary data of the monitoring data by cubic spline interpolation.

Optionally, the collecting multimodal data of said target object includes:
pushing a questionnaire targeting the preset symptoms to the target object;
receiving survey data input by the target subject in response to the questionnaire, where the survey data includes a selection input of at least one option among a plurality of options for each question set for the preset symptom;
standardizing the survey data according to preset rules to form multimodal data, where the standardized survey data are used as external variables of a health assessment model.

A health assessment method is provided in the embodiment of the present disclosure, including:
acquiring physical state data of the target object, where the physical state data is obtained by the data processing method hereinabove;
inputting the physical condition data into a health assessment model to obtain a health assessment result of the target object suffering from a target disease, where the health assessment model is a pre-trained model that takes the physical condition data as input and the probability of suffering from the target disease as output.

Optionally, the health assessment model includes an integrated first model, a second model and a third model, where the first model is an ARIMA model, the second model is an Informer model, and the third model is an N-BeatXs model.

Optionally, the input data of the first model and the second model include the vital sign data;
the input data of the third model includes the vital sign data and the multimodal data.

Optionally, the health assessment model further includes a fourth model, where the fourth model is a model which takes the resampled vital sign data as input and takes the probability of suffering from the target disease as output.

Optionally, the fourth model is an Informer model.

Optionally, the step of obtaining a health assessment result of the target subject suffering from the target disease by using the health assessment model includes:
resampling the vital sign data into high-frequency data and low-frequency data, where the sampling frequency of the high-frequency data is greater than the sampling frequency of the low-frequency data, and the sampling frequency of the low-frequency data is no less than twice in each monitoring cycle;
performing difference processing on the low-frequency data according to the sampling frequency of the high-frequency data;
inputting the high-frequency data and the low-frequency data after difference processing respectively into the fourth model to obtain the prediction result of the periodic trend of the vital sign data.

Optionally, inputting the physical state data into a health assessment model to obtain a health assessment result of the target subject suffering from a target disease includes:

splicing the output results of the first model, the second model, the third model and the third model in time and input into the fifth model for integrated training to obtain the health assessment model.

Optionally, the fifth model is a LightGBM model.

Optionally, the target disease is chronic obstructive pulmonary disease.

A data processing device is provided in the embodiment of the present disclosure, including:
a vital sign data acquisition module, configured to acquire vital sign data of a target object, where the vital sign data includes monitoring data obtained by monitoring the vital signs of the target object and supplemented data supplemented by the vital sign data according to the monitoring data;
a multimodal data acquisition module, configured to acquire multimodal data of the target object, where the multimodal data includes at least one of image data of the target object and survey data for preset symptoms; and
a physical state data generating module, configured to generate the physical state data of the target object according to the vital sign data and the multimodal data.

A health assessment device is provided in the embodiment of the present disclosure, including:
a physical state data acquisition module, configured to acquire physical state data of a target object, where the physical state data is obtained by the data processing method hereinabove;
a health assessment module, configured to input the physical condition data into a health assessment model to obtain a health assessment result of the target object suffering from a target disease, where the health assessment model is a pre-trained model that takes the physical condition data as input and the probability of suffering from the target disease as output.

An electronic device is provided in the embodiment of the present disclosure, including: a memory, a processor, and a program stored in the memory and executable on the processor; the processor is configured to read the program in the memory to perform the steps of the method hereinabove.

A readable storage medium is provided in the embodiment of the present disclosure, for storing a program, where when the program is executed by a processor, the steps in the method in the first aspect or the second aspect hereinabove is performed.

### BRIEF DESCRIPTION OF THE DRAWINGS

In order to more clearly illustrate the technical solutions of the embodiments of the present disclosure, the drawings required for use in the description of the embodiments of the present disclosure will be briefly introduced below. Obviously, the drawings described below are only some embodiments of the present disclosure. For ordinary technicians in this field, other drawings can be obtained based on these drawings without paying creative labor.

FIG. 1 is a flow chart of a data processing method provided by an embodiment of the present disclosure;

FIG. 2A is a schematic diagram of a questionnaire provided by an embodiment of the present disclosure;

FIG. 2B is a schematic diagram of another questionnaire provided by an embodiment of the present disclosure;

FIG. 2C is a schematic diagram of another questionnaire provided by an embodiment of the present disclosure;

FIG. 3 is a flow chart of a health assessment process provided by an embodiment of the present disclosure;

FIG. 4 is a schematic diagram of a model architecture provided by an embodiment of the present disclosure;

FIG. 5 is a schematic diagram of another model architecture provided by an embodiment of the present disclosure;

FIG. 6 is a schematic diagram of another model architecture provided by an embodiment of the present disclosure;

FIG. 7 is a schematic diagram of the structure of a data processing device provided by an embodiment of the present disclosure; and

FIG. 8 is a schematic diagram of the structure of an electronic device provided by an embodiment of the present disclosure.

### DETAILED DESCRIPTION

The following will be combined with the drawings in the embodiments of the present disclosure to clearly and completely describe the technical solutions in the embodiments of the present disclosure. Obviously, the described embodiments are part of the embodiments of the present disclosure, rather than all of the embodiments. Based on the embodiments in the present disclosure, all other embodiments obtained by ordinary technicians in this field without creative work are within the scope of protection of the present disclosure.

The terms “first” and “second” and the like in the disclosed embodiments are used to distinguish similar objects, and need not be used to describe a specific order or sequential order. In addition, the terms “include” and “have” and any variation thereof are intended to cover non-exclusive inclusions, for example, the process, method, system, product or equipment comprising a series of steps or units need not be limited to those steps or units clearly listed, but may include other steps or units that are not clearly listed or inherent to these processes, methods, products or equipment. In addition, “and/or” is used in the present application to represent at least one of the connected objects, such as A and/or B and/or C, indicating that A alone, B alone, C alone, and A and B all exist, B and C all exist, A and C all exist, and 7 situations in which A, B and C all exist.

An embodiment of the present disclosure provides a data processing method.

In some of the embodiments, the data processing method is used to collect and process the collected physical state data of the user to obtain more comprehensive and complete data.

Taking patients with chronic obstructive pulmonary disease (COPD) as an example, theoretically, if you need to obtain the patient's complete physical condition data, you need to monitor the patient's physical condition for several consecutive days for 24 hours. The data that needs to be monitored includes various data such as dynamic electrocardiogram, continuous blood sugar, blood oxygen saturation, etc. However, this kind of continuous monitoring for several consecutive days is difficult to achieve. Therefore, in actual operation, physical signs are usually monitored during specific time periods.

For example, blood oxygen saturation may be measured for ten minutes at eight in the morning, half an hour at three in the afternoon, and continuously monitored for 4 to 5 hours at night. If data sampling is performed at one-minute intervals, 10 data items can be obtained at eight in the morning, 30 data items at three in the afternoon, and 240 to 300 data items can be obtained at night. However, when the patient feels unwell, it may be necessary to measure changes in physical condition at any time, and these irregular intermittent data cannot be used directly.

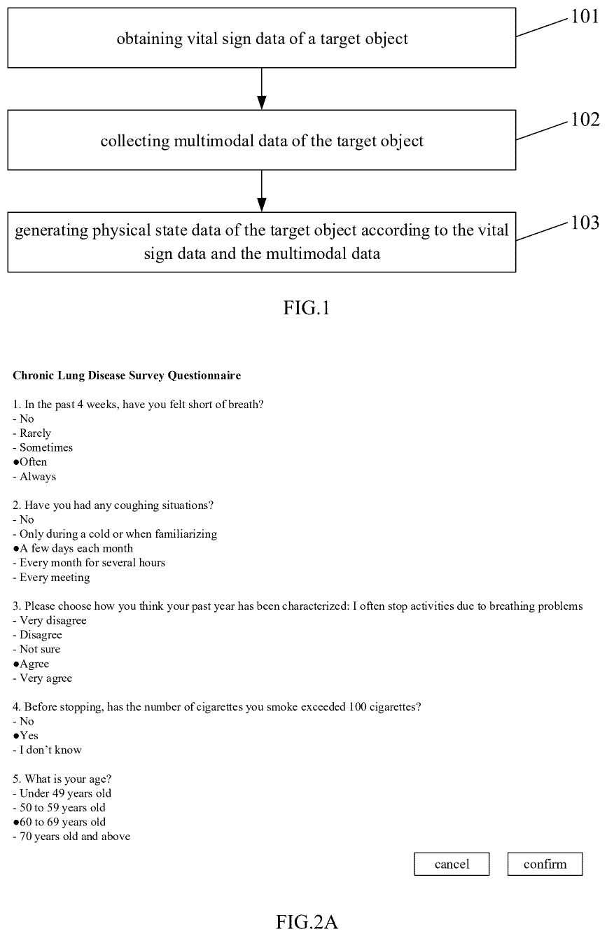

As shown in FIG. 1, in one embodiment of the present disclosure, the data processing method includes the following steps:

Step 101: obtaining vital sign data of a target object.

In the technical solution of this embodiment, the vital sign data includes two parts: monitoring data and supplementary data, wherein the monitoring data refers to the monitoring of the vital sign data of the target object (for example, it can be a patient) according to certain measurement conditions. Exemplarily, the monitoring time can be set to monitor the patient's physical condition at a specified time or time period every day. For example, the blood oxygen saturation can be measured for 10 minutes at 8 o'clock in the morning, the blood oxygen saturation can be measured for 30 minutes at 3 o'clock in the afternoon, and the blood oxygen saturation can be continuously monitored for 4 to 5 hours at night. Specific items of monitoring data may include blood oxygen saturation, heart rate, dynamic electrocardiogram, continuous blood sugar, etc. During implementation, the specific items to be detected can be determined by professional doctors and other professionals according to the type of disease to be detected or predicted.

During implementation, when the target object is undergoing quality and normal respiratory sleep monitoring, monitoring data of the target object can be collected using detection instruments such as a ventilator, a pulmonary function meter, a blood oximeter, a respiratory detector, etc., and then the data is collected and processed to form the monitoring data of the target object.

After obtaining the monitoring data obtained by monitoring the target object, the obtained monitoring data is supplemented to obtain supplemented data to improve the integrity of the obtained vital sign data. It should be understood that continuous monitoring of the vital signs of the target object is difficult to implement in practice. In this embodiment, the monitoring data is supplemented to obtain supplemented data, and the monitoring data and the monitoring data are used as the vital sign data of the target object. It is possible to obtain more accurate and comprehensive physical data of the target object through limited monitoring means and monitoring time, thereby improving the data collection effect.

In some embodiments, the step of completing the data further includes:
acquiring monitoring data obtained by monitoring the vital signs of the target object;
binning the monitoring data;
grouping the monitoring data after binning according to a preset monitoring period;
generating supplementary data to supplement the missing monitoring data in each monitoring cycle;
using the monitoring data and the supplemented data as vital sign data of the target object.

In this embodiment, by binning the monitoring data and then grouping them according to the monitoring period, and further completing the monitoring data in each monitoring period after binning, it can be ensured that when the data is completed, the data in each bin has a high correlation, thereby improving the accuracy of the completed data.

In this embodiment, the monitoring data is first divided into boxes. In some embodiments, the step of dividing the monitoring data into boxes specifically includes:

The monitoring data is binned according to the degree of impact of the monitoring data on the target disease.

In the technical solution of this embodiment, the monitoring data is obtained by monitoring the physical signs of the target object, and different monitoring results may reflect different possibilities of the target object suffering from the target disease. For example, taking chronic obstructive pulmonary disease as an example, under the same other conditions, the lower the blood oxygen concentration of the target object and the higher the heart rate, the higher the possibility of illness and the higher the symptom level.

In order to distinguish such influence, in this embodiment, the monitoring data is binned by the interval size of the influence degree of different monitoring data on the target disease. It can be understood that, under the same other conditions, if the difference in the heart rate of the two target objects is 1, it may be a normal fluctuation and has no medical or clinical reference significance. If the difference reaches 5 or 10, it may have certain medical or clinical reference significance. Therefore, in order to distinguish different monitoring data, in this embodiment, the monitoring data can be binned according to the medical or clinical reference significance of the difference in the monitoring results for the influence degree of the target disease. It can be understood that if a certain interval corresponding to the heart rate has the same level for the target disease, the monitoring data will be divided into data in one bin.

In this way, by binning the monitoring data according to the degree of influence of the monitoring data on the target disease, the monitoring data can be accurately divided, and the subsequent completion of the binned data can improve the accuracy of the obtained data and improve the diagnostic effect of the target disease.

Furthermore, in one of the embodiments, the monitoring data may be binned using a minimum entropy binning method.

In this embodiment, the binning results of the minimum entropy binning method need to meet the following requirements:

$$arg min ( - {\sum }_{i = 0}^{K} {\sum }_{j = 0}^{J} ( {p}_{ij} \times log {p}_{ij} ) ) ;$$

The dependent variable is a categorical variable and can take values 1, 2, 3 . . . J. pᵢⱼ is the proportion of observations in the i-th bin whose dependent variable takes the value j, where i=1, 2, 3, . . . K and j=1, 2, 3, . . . J.

During implementation, the monitoring data set is first sorted and counted. Next, the monitoring data is divided into two, and the entropy of the two subsets is calculated and then summed. Next, the division point with the smallest sum of conditional entropy is selected, and then for each subset obtained, the above steps of sorting, dividing, and summing conditional entropy are repeated until the condition for stopping iteration is met.

In this embodiment, the condition for stopping the iteration can be determined according to the advice of professionals such as doctors. For example, if the doctor indicates that the heart rate difference within 5 times is clinically significant, the condition for stopping the iteration can be set to that the interval length of the obtained subset is less than or equal to 5. In this way, this embodiment can more accurately extract the differences between the monitoring data by binning the monitoring data using the minimum entropy binning method, thereby obtaining a more accurate binning result for the monitoring data, which helps to improve the accuracy of subsequent data completion.

After the data is binned, it is divided into multiple groups according to a certain monitoring period. Generally speaking, the selection of the monitoring period needs to have certain practical significance. Therefore, in this embodiment, each day is used as a monitoring period. In this way, under normal circumstances, within each monitoring period, the vital signs of the target object change from morning to night according to a certain rule.

In some embodiments, after grouping the monitoring data after binning according to a preset monitoring period, the method further includes:
detecting the number of first cycles in the monitoring cycle, wherein the first cycle is a monitoring cycle in which monitoring data at a target time is missing;
when the number of the first cycles is greater than a preset number threshold, the monitoring data at the target moment of the first cycle is determined according to the monitoring data at the target moment of the second cycle, wherein the second cycle is a monitoring period in which the monitoring data at the target moment is not missing.

In this embodiment, the number of monitoring cycles with missing data is further detected. Exemplarily, if most of the multiple monitoring cycles obtained include heart rate data at 8 o'clock, and the heart rate data at 8 o'clock is missing in several of the detection cycles, the monitoring cycle with missing heart rate data at 8 o'clock is defined as the first cycle, and the monitoring cycle with no missing heart rate data at 8 o'clock is defined as the second cycle.

Next, determine the number of first cycles. If the number of first cycles is less than a preset number threshold, exemplarily, there is only one first cycle, then the absence of heart rate data at 8 o'clock in the first cycle is considered to be accidental. If the number of first cycles is large, then it is considered that there is an abnormality in the number of first cycles. At this time, it is necessary to compensate for the monitoring data of the target moment in the first cycle. During implementation, the monitoring data of the target moment in the first cycle can be compensated by weighted averaging based on the monitoring data of the target moment in the second cycle.

In some embodiments, the generating of the supplementary data for supplementing the missing monitoring data in each monitoring cycle includes:

The complementary data of the monitoring data are generated by cubic spline interpolation.

In this embodiment, for the missing data to be completed, it needs to be understood that the changes in human vital signs data are smooth. Therefore, under normal circumstances, the human vital signs data will not experience sudden changes, that is, when reflected on the curve, the corresponding human vital signs data curve will not have a sharp inflection point. Therefore, linear interpolation is not used for data completion in this embodiment.

Polynomial interpolation increases, the amount of calculation increases greatly, and oscillation (Runge phenomenon) may occur near the endpoints. Therefore, a piecewise low-order interpolation method is adopted in this embodiment. Specifically, cubic spline interpolation is adopted to construct multiple cubic functions from several segments in the original monitoring data sequence, so that each segment connection has the property of continuous second-order derivatives and can be smoothly connected, which balances the amount of calculation and improves the accuracy of the completed data.

It should be understood that before data completion, the acquired monitoring data may be data accumulated over a long period of time, for example, multiple segments of data within a few months or years, each segment of data being separated by the same time period (for example, 1 minute). Therefore, in this embodiment, after completion, the data between two adjacent segments is separated by this time period, forming a curve without interruption.

During implementation, the monitoring data of each target object can be used as a sample, and the value of the blood oxygen saturation and other monitoring indicators can be used as labels to form a data set of vital sign data corresponding to each target object.

Step 102: collecting multimodal data of the target object.

Next, in this embodiment, multimodal data of the target object is extracted. In one embodiment, the multimodal data includes at least one of image data of the target object and survey data for preset symptoms.

In this embodiment, the image data refers to the analysis results of detection images obtained by various imaging detection methods such as CT Computed Tomography), X-ray, and magnetic resonance imaging.

Taking the image data as the analysis result of CT images as an example, in clinical practice, COPD is generally divided into four levels. Therefore, in this embodiment, the analysis results of CT images are set to five types, corresponding to negative (not sick) and four different levels of symptoms. In an exemplary embodiment, the relatively mature ResNet151 model can be used to identify and process the obtained CT images, and its output range is defined as 0 to 4, where 0 corresponds to negative, and 1 to 4 correspond to four different levels of symptoms. In this way, the image data of the target object can be obtained.

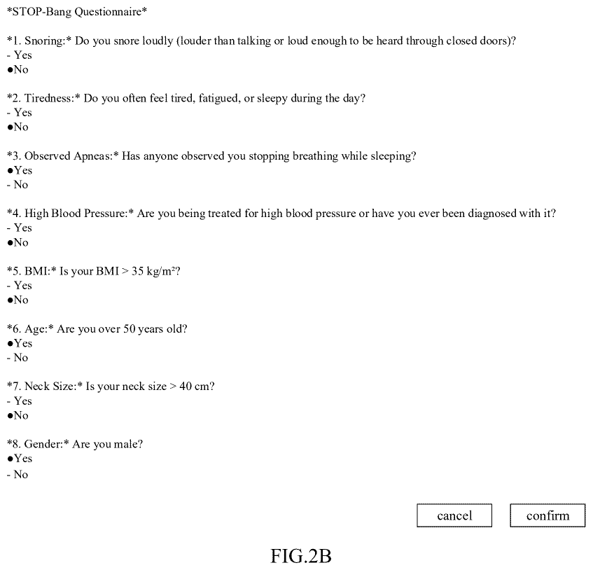

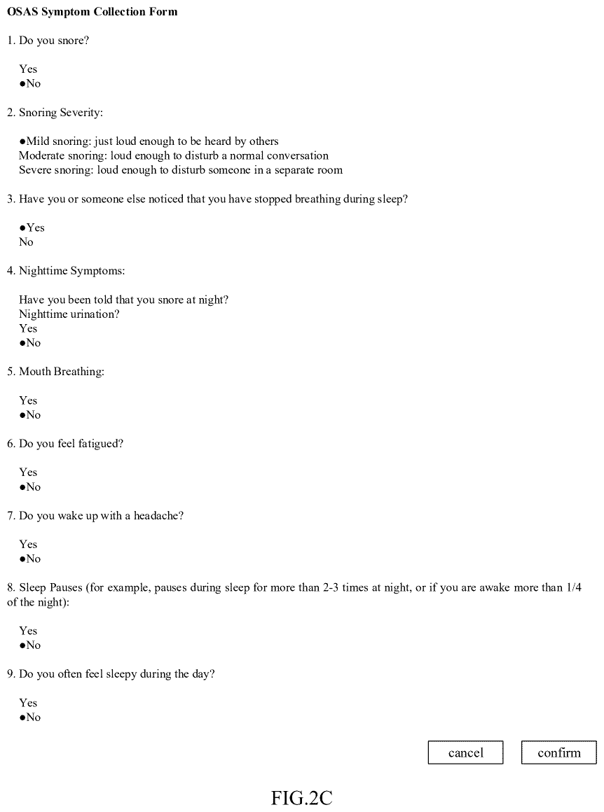

The survey data can be set up with corresponding questionnaires for different target diseases. For example, as shown in FIGS. 2A to 2C, in this embodiment, a COPD screening questionnaire as shown in FIG. 2A, a STOP-Bang questionnaire for determining risk factors for obstructive sleep apnea, and an OSAS symptom collection table for determining risk factors for obstructive sleep apnea syndrome are set up for COPD. During implementation, doctors and other professionals can set up corresponding questionnaires for different preset symptoms to generate survey data for the preset symptoms.

FIG. 2A to FIG. 2C, in this embodiment, a questionnaire can be pushed to the target subject's account, and the target subject can log in to his/her account on a mobile terminal such as a mobile phone or a personal computer, or a diagnosis and treatment terminal in a hospital or health care facility, and fill in the corresponding questionnaire. In other embodiments, corresponding equipment can also be set to collect corresponding data, such as monitoring the snoring volume through a sound sensor. In addition, a nurse or a monitor can monitor the target subject's sleep state while he/she is sleeping and fill in the corresponding data.

The questionnaire results of the target object are standardized according to certain rules and sent to the host side as multimodal data corresponding to the target object.

During implementation, the quantitative indicators of the survey results based on the questionnaires filled out by the target subjects are used as research data.

Step 103: generating physical state data of the target object according to the vital sign data and the multimodal data.
after the vital sign data and the multimodal data are determined, the obtained vital sign data and the multimodal data are used as the physical state data of the target object.

The disclosed embodiment also provides a health assessment method.

As shown in FIG. 3, in one embodiment, the health assessment method includes the following steps:

Step 301: obtaining the physical state data of the target object.

In this embodiment, the physical state data of the target object is first obtained, wherein the physical state data is obtained by the above-mentioned data processing method, which will not be described in detail here.

Step 302: inputting the physical condition data into a health assessment model to obtain a health assessment result of the target object suffering from the target disease, wherein the health assessment model is a pre-trained model that takes the physical condition data as input and the probability of suffering from the target disease as output.

In this embodiment, by setting up a pre-trained health assessment model, the prediction results of the target object's illness can be obtained conveniently and quickly based on the input physical data, which reduces manual participation and saves human resources. It can accurately and quickly generate preliminary prediction results of the target object's illness, and can provide important reference information for the target object to seek medical treatment or doctor's diagnosis.

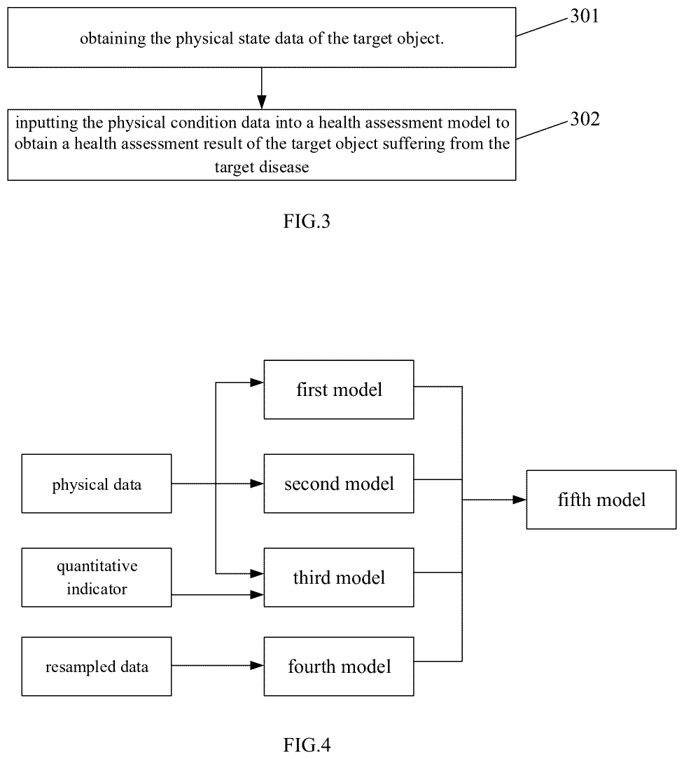

As shown in FIG. 4, in some embodiments, the health assessment model includes an integrated first model, a second model, and a third model. In some embodiments, the first model is an ARIMA model, the second model is an Informer model, and the third model is an N-BeatXs model. In this embodiment, the input data of the first model and the second model include vital sign data, as shown in FIG. 5, the input data of the third model includes vital sign data and multimodal data, and further, the multimodal data includes imaging data and survey data.

It should be understood that the ARIMA model is a time series model in traditional machine learning. It has a better prediction effect on short-term weak stationary series. The Informer model has better prediction results for long periods of time. It is based on the characteristics of time series. On the basis of the Transformer model, it adds self-attention distillation mechanism and generative Decoder and other optimization schemes, which greatly improves the complexity and efficiency of time series prediction. In addition, in this embodiment, the data of quantitative indicators is input into the N-BeatsX model. The N-BeatsX model can input time series data and external variables at the same time, because from a medical point of view, the impact of external characteristics on diseases is very important. The N-BeatXs model can capture the impact of other factors.

In the technical solution of this embodiment, multiple integrated models are set up to process the physical condition data of the target object, and different characteristics of different models are used to obtain more accurate health assessment results for the target disease.

As shown in FIG. 4, in some embodiments, the health assessment model further includes a fourth model, which is a model that takes the resampled vital sign data as input and takes the probability of the target disease as output. In some embodiments, the fourth model is an Informer model.

In some embodiments, obtaining a health assessment result of the target subject suffering from the target disease by the health assessment model includes:
resampling the vital sign data into high-frequency data and low-frequency data, wherein the sampling frequency of the high-frequency data is greater than the sampling frequency of the low-frequency data, and the sampling frequency of the low-frequency data is no less than twice in each monitoring cycle;
performing difference processing on the low-frequency data according to the sampling frequency of the high-frequency data;
the high-frequency data and the low-frequency data after difference processing are respectively input into the fourth model to obtain the prediction result of the periodic trend of the vital sign data.

In this embodiment, the monitoring data is resampled into low-frequency data and high-frequency data, and the resampled monitoring data are respectively input into the fourth model.

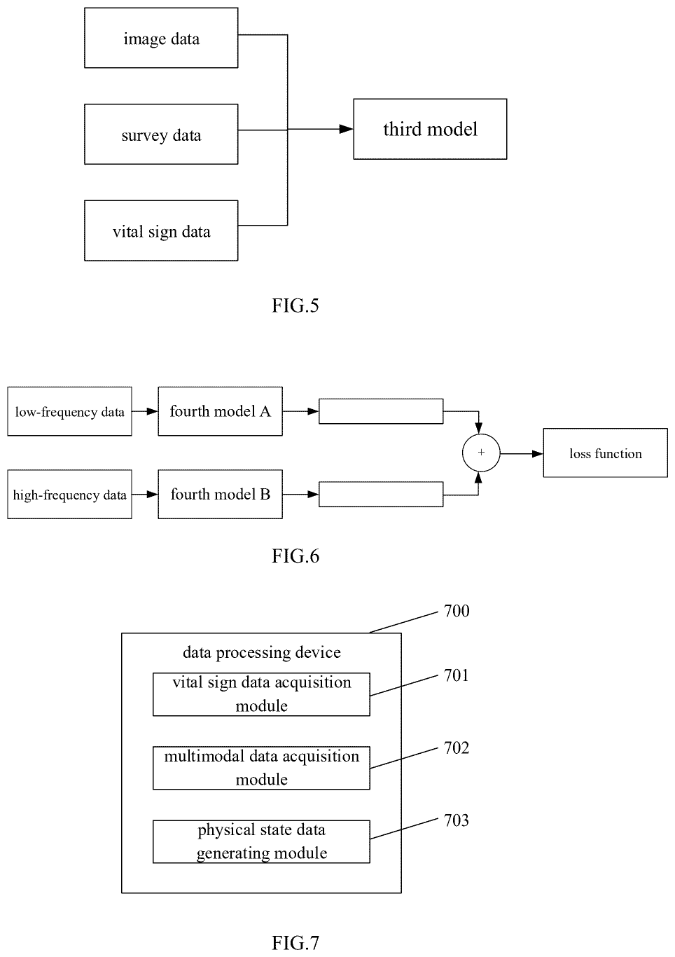

As shown in FIG. 6, in this embodiment, the fourth model specifically includes a fourth model A for processing low-frequency data and a fourth model B for processing high-frequency data.

In order to enable the low-frequency data model to analyze periodic changes, according to Shannon's theorem, if the low-frequency sampled data wants to obtain periodic changes, it should be sampled at a sampling frequency of not less than twice a day. After down-sampling to obtain low-frequency data, in order to add it to the high-frequency data, the low-frequency data is interpolated using the same frequency as the high-frequency data. In this embodiment, piecewise linear interpolation is specifically selected.

In this embodiment, the outputs of the fourth model are all classification probabilities of five categories, corresponding to 0 to 4 mentioned above. The low-frequency data and the high-frequency data are added through the model respectively and then calculated. The loss is calculated using the cross entropy loss function. During optimization, the loss is simultaneously passed back to the fourth model A corresponding to the low-frequency data and the fourth model B corresponding to the high-frequency data.

In some embodiments, inputting the physical state data into a health assessment model to obtain a health assessment result of the target subject suffering from a target disease includes:

The output results of the first model, the second model, the third model and the fourth model are spliced in time, and then input into the fifth model for integrated training to obtain the health assessment model.

In some of the embodiments, the fifth model is a LightGBM model. In this embodiment, the output results of the first model, the second model, the third model and the fourth model are finally input into the fifth model for integrated training, and the model obtained by training is used as a health assessment model as a whole.

In some of the embodiments, during the integrated training process, the parameters of the first model, the second model, the third model and the fourth model are frozen so that they no longer change, and then the original data are input in batches, and the output data of these models are spliced and given to the LightGBM model. The output is the probability of the five categories from 0 to 4 mentioned above. The cross entropy is used as the loss function, and the SGD stochastic gradient descent method is used as the optimization method for integrated training to obtain a health assessment model.

In the technical solution of this embodiment, the output result of the generated health assessment model can be a health assessment result of five levels from 0 to 4, where 0 represents negative, no disease, and 1 to 4 represent different disease severity levels.

During implementation, the above-mentioned disease level can be pushed to users in the form of examination results, diagnosis sheets, etc. In order to facilitate users to understand their disease level, a detailed introduction or description of the disease level can be added to the push results, and corresponding medical advice can be pushed at the same time, such as adding various additional information such as eating habits, living habits, and smoking cessation suggestions.

In some other embodiments, the prediction results may also be sent to professionals, such as doctors, who refer to the prediction results to form medical advice or treatment results, and push them to the target object together with additional information such as corresponding treatment methods.

The embodiment of the present disclosure also provides a data processing device.

As shown in FIG. 7, in one embodiment, the data processing device 700 includes:
a vital sign data acquisition module 701, configured to acquire vital sign data of a target object, wherein the vital sign data includes monitoring data obtained by monitoring the vital signs of the target object and supplemented data obtained by supplementing the vital sign data according to the monitoring data;
a multimodal data acquisition module 702, configured to acquire multimodal data of the target object, wherein the multimodal data includes at least one of image data of the target object and survey data for preset symptoms;
a physical state data generating module 703, configured to generate the physical state data of the target object according to the vital sign data and the multimodal data.

In some embodiments, the vital sign data acquisition module 701 includes:
an acquisition submodule, used to acquire monitoring data obtained by monitoring the vital signs of the target object;
a binning module, used for binning the monitoring data;
a grouping submodule, used for grouping the monitoring data after binning according to a preset monitoring period;
a completion submodule, used to generate completion data to complete the missing monitoring data in each monitoring cycle;

The vital sign data confirmation submodule is used to use the monitoring data and the supplemented data as the vital sign data of the target object.

In some embodiments, the binning module is specifically used to bin the monitoring data according to the degree of impact of the monitoring data on the target disease.

In some embodiments, the binning module is specifically used to bin the monitoring data using a minimum entropy binning method.

In some embodiments, it also includes:
a monitoring cycle detection module, used to detect the number of first cycles in the monitoring cycle, wherein the first cycle is a monitoring cycle in which monitoring data at a target time is missing;
a monitoring data determination module is used to determine the monitoring data of the target moment of the first cycle based on the monitoring data of the target moment of the second cycle when the number of the first cycle is greater than a preset number threshold, wherein the second cycle is a monitoring period in which the monitoring data at the target moment is not missing.

In some embodiments, the completion submodule is specifically used to generate the completion data of the monitoring data by cubic spline interpolation.

In some embodiments, the multimodal data acquisition module 702 includes:
a questionnaire push submodule is used to push a questionnaire targeting the preset symptoms to the target object;
a survey data receiving submodule, configured to receive survey data input by the target subject for the questionnaire, wherein the survey data includes a selection input of at least one option among a plurality of options for each question set for the preset symptom;

The multimodal data generation submodule is used to standardize the survey data according to preset rules to form multimodal data, wherein the standardized survey data is used as an external variable of the health assessment model.

This embodiment can implement each step of the above-mentioned data processing method embodiment and can achieve basically the same technical effects, which will not be described in detail here.

The present disclosure also provides a health assessment device, including:
a physical state data acquisition module, used to acquire physical state data of a target object, wherein the physical state data is obtained by the data processing method described in any one of the first aspects;
a health assessment module, used to input the physical condition data into a health assessment model to obtain a health assessment result of the target object suffering from a target disease, wherein the health assessment model is a pre-trained model that takes the physical condition data as input and the probability of suffering from the target disease as output.

In some embodiments, the health assessment model includes an integrated first model, a second model, and a third model, wherein the first model is an ARIMA model, the second model is an Informer model, and the third model is an N-BeatXs model.

In some embodiments, the input data of the first model and the second model include the vital sign data;
the input data of the third model includes the vital sign data and the multimodal data.

In some embodiments, the health assessment model also includes a fourth model, which is a model that takes the resampled vital sign data as input and takes the probability of suffering from the target disease as output.

In some embodiments, the fourth model is an Informer model.

In some embodiments, the health assessment module comprises:
a resampling submodule, used for resampling the vital sign data into high-frequency data and low-frequency data, wherein the sampling frequency of the high-frequency data is greater than the sampling frequency of the low-frequency data, and the sampling frequency of the low-frequency data is not less than twice in each monitoring cycle;
a difference processing submodule, used for performing difference processing on the low-frequency data according to the sampling frequency of the high-frequency data;
the input submodule is used to input the high-frequency data and the low-frequency data after difference processing into the fourth model respectively to obtain the prediction result of the periodic trend of the vital sign data.

In some embodiments, the health assessment module is specifically used to splice the output results of the first model, the second model, the third model and the third model in time, and input them into the fifth model for integrated training to obtain the health assessment model.

In some embodiments, the fifth model is a LightGBM model.

In some embodiments, the target disease is chronic obstructive pulmonary disease.

This embodiment can implement each step of the above-mentioned health assessment method embodiment and can achieve basically the same technical effects, which will not be repeated here.

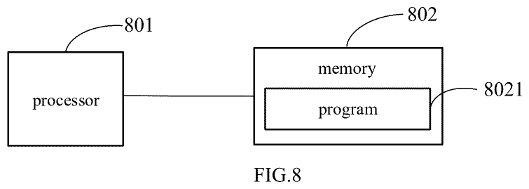

The embodiment of the present disclosure further provides an electronic device. Referring to FIG. 8, the electronic device may include a processor 801, a memory 802, and a program 8021 stored in the memory 802 and executable on the processor 801.

When program 8021 is executed by processor 801, any steps in the above method embodiment can be implemented and the same beneficial effects can be achieved, which will not be described in detail here.

Those skilled in the art will appreciate that all or part of the steps of implementing the above-mentioned embodiment method can be completed by hardware associated with program instructions, and the program can be stored in a readable medium.

The embodiments of the present disclosure also provide a readable storage medium having a computer program stored thereon. When the computer program is executed by a processor, any step in the above method embodiment can be implemented and the same technical effect can be achieved. To avoid repetition, it will not be described here.

The storage medium may be a read-only memory (ROM), a random access memory (RAM), a magnetic disk or an optical disk.

It should be noted that it should be understood that the division of the above modules is only a division of logical functions. In actual implementation, they can be fully or partially integrated into one physical entity, or they can be physically separated. And these modules can all be implemented in the form of software called by processing elements; they can also be all implemented in the form of hardware; some modules can also be implemented in the form of software called by processing elements, and some modules can be implemented in the form of hardware. For example, the determination module can be a separately established processing element, or it can be integrated in a chip of the above-mentioned device for implementation. In addition, it can also be stored in the memory of the above-mentioned device in the form of program code, and called and executed by a processing element of the above-mentioned device. The implementation of other modules is similar. In addition, these modules can be fully or partially integrated together, or they can be implemented independently. The processing element described here can be an integrated circuit with signal processing capabilities. In the implementation process, each step of the above method or each module above can be completed by an integrated logic circuit of hardware in the processor element or instructions in the form of software.

For example, each module, unit, sub-unit or sub-module may be one or more integrated circuits configured to implement the above method, such as one or more application specific integrated circuits (ASIC), or one or more digital signal processors (DSP), or one or more field programmable gate arrays (FPGA). For another example, when a module above is implemented in the form of a processing element scheduling program code, the processing element may be a general-purpose processor, such as a central processing unit (CPU) or other processor that can call program code. For another example, these modules may be integrated together and implemented in the form of a system-on-a-chip (SOC).

The above is a preferred implementation of the embodiment of the present disclosure. It should be pointed out that for ordinary technicians in this technical field, several improvements and modifications can be made without departing from the principles described in the present disclosure. These improvements and modifications should also be regarded as the scope of protection of the present disclosure.

1. A data processing method, comprising:
acquiring vital sign data of a target object, wherein the vital sign data comprises monitoring data obtained by monitoring vital signs of the target object and supplemented data obtained by supplementing the vital sign data according to the monitoring data;
collecting multimodal data of the target object, wherein the multimodal data comprises at least one of image data of the target object and survey data for preset symptoms; and
generating physical state data of the target object according to the vital sign data and the multimodal data.

2. The method according to claim 1, wherein the obtaining the vital sign data of the target object comprises:
acquiring monitoring data obtained by monitoring the vital signs of the target object;
binning the monitoring data;
grouping the monitoring data after binning according to a preset monitoring period;
generating supplementary data to supplement the missing monitoring data in each monitoring cycle; and
using the monitoring data and the supplemented data as the vital sign data of the target object.

3. The method according to claim 2, wherein the binning the monitoring data comprises: binning the monitoring data according to a degree of impact of the monitoring data on the target disease.

4. The method according to claim 3, wherein the binning the monitoring data comprises: binning the monitoring data using the minimum entropy binning method.

5. The method according to claim 2, wherein after the monitoring data after binning is grouped according to a preset monitoring period, the method further comprises:
detecting a number of first cycles in the monitoring cycle, wherein the first cycle is a monitoring cycle in which monitoring data at a target time is missing;
when the number of the first cycles is greater than a preset number threshold, determining the monitoring data at the target moment of the first cycle according to the monitoring data at the target moment of the second cycle, wherein the second cycle is a monitoring period in which the monitoring data at the target moment is not missing.

6. The method according to claim 2, wherein the generating the supplementary data for supplementing the missing monitoring data in each monitoring cycle comprises:
generating the complementary data of the monitoring data by cubic spline interpolation.

7. The method according to claim 1, wherein the collecting multimodal data of the target object comprises:
pushing a questionnaire targeting the preset symptoms to the target object;
receiving survey data input by the target subject in response to the questionnaire, wherein the survey data comprises a selection input of at least one option among a plurality of options for each question set for the preset symptom;
standardizing the survey data according to preset rules to form the multimodal data, wherein the standardized survey data are used as external variables of a health assessment model.

8. A health assessment method, comprising:
acquiring physical state data of the target object, wherein the physical state data is obtained by the data processing method according to claim 1;
inputting the physical condition data into a health assessment model to obtain a health assessment result of the target object suffering from a target disease, wherein the health assessment model is a pre-trained model that takes the physical condition data as input and the probability of suffering from the target disease as output.

9. The method according to claim 8, wherein the health assessment model comprises an integrated first model, a second model and a third model, wherein the first model is an ARIMA model, the second model is an Informer model, and the third model is an N-BeatXs model.

10. The method according to claim 9, wherein the input data of the first model and the second model comprise the vital sign data;
the input data of the third model comprises the vital sign data and the multimodal data.

11. The method according to claim 9, wherein the health assessment model further comprises a fourth model, wherein the fourth model is a model which takes the resampled vital sign data as input and takes the probability of suffering from the target disease as output.

12. The method according to claim 11, wherein the fourth model is an Informer model.

13. The method according to claim 11, wherein the obtaining a health assessment result of the target subject suffering from the target disease by using the health assessment model comprises:
resampling the vital sign data into high-frequency data and low-frequency data, wherein a sampling frequency of the high-frequency data is greater than the sampling frequency of the low-frequency data, and a sampling frequency of the low-frequency data is no less than twice in each monitoring cycle;
performing difference processing on the low-frequency data according to the sampling frequency of the high-frequency data; and
inputting the high-frequency data and the low-frequency data after difference processing respectively into the fourth model to obtain the prediction result of the periodic trend of the vital sign data.

14. The method according to claim 11, wherein inputting the physical state data into a health assessment model to obtain a health assessment result of the target subject suffering from a target disease comprises:
splicing output results of the first model, the second model, the third model and the third model in time and input into the fifth model for integrated training to obtain the health assessment model.

15. The method according to claim 14, wherein the fifth model is a LightGBM model.

16. The method according to claim 8, wherein the target disease is chronic obstructive pulmonary disease.

17. A data processing device, comprising:
a vital sign data acquisition module, configured to acquire vital sign data of a target object, wherein the vital sign data includes monitoring data obtained by monitoring the vital signs of the target object and supplemented data supplemented by the vital sign data according to the monitoring data;
a multimodal data acquisition module, configured to acquire multimodal data of the target object, wherein the multimodal data includes at least one of image data of the target object and survey data for preset symptoms; and
a physical state data generating module, configured to generate the physical state data of the target object according to the vital sign data and the multimodal data.

18. A health assessment device, comprising:
a physical state data acquisition module, configured to acquire physical state data of a target object, wherein the physical state data is obtained by the data processing method according to claim 1;
a health assessment module, configured to input the physical condition data into a health assessment model to obtain a health assessment result of the target object suffering from a target disease,
wherein the health assessment model is a pre-trained model that takes the physical condition data as input and the probability of suffering from the target disease as output.

19. An electronic device, comprising: a memory, a processor, and a program stored in the memory and executable on the processor; the processor is configured to read the program in the memory to perform the steps of the method according to claim 1.

20. A readable storage medium, storing a program, wherein when the program is executed by a processor, the steps in the method according to claim 1.
